(19) United States
(12) Patent Application Publication
(10) Pub. No.: US 2018/0028571 A1
(43) Pub. Date: Feb. 1, 2018

(54) METHOD FOR TREATING CANCER

(71) Applicants: UNIVERSITE PARIS DESCARTES, Paris Cedex 06 (FR); Assistance Publique - Hopitaux De Paris, Paris (FR); INSERM, Paris (FR); Ministere De La Defense Service De Sante Des Armees, Clamart (FR)

(72) Inventors: Bruno GOGLY, Hondevilliers (FR); Antoine LAFONT, Paris (FR); Bernard COULOMB, Igny (FR); Jean-Jacques LATAILLADE, Elancourt (FR)

(21) Appl. No.: 15/782,345

(22) Filed: Oct. 12, 2017

Related U.S. Application Data
(63) Continuation of application No. 15/046,190, filed on Feb. 17, 2016.
Application No. 15/046,190 is a continuation of application No. 13/255,039, filed on Dec. 16, 2011, now abandoned.
(60) Provisional application No. 61/158,037, filed on Mar. 6, 2009.

(51) Int. Cl. C12N 5/09; A61K 35/12

(52) U.S. Cl. CPC A61K 35/33; C12N 5/0693; A61K 35/12; C12N 2502/1323; C12N 2502/097

(57) ABSTRACT

The present invention relates to a method for preventing or treating cancer in an individual comprising administering the individual with a prophylactically or therapeutically effective quantity of a gingival fibroblast-derived product.

### FIELD OF THE INVENTION

The present invention relates to a method for treating cancer, in particular by inhibiting tumor invasion.

### BACKGROUND OF THE INVENTION

Tumor invasion is a key step in cancer progression, in which malignant cells with high invasive potential diffuse trough the basal lamina and form metastases. Accordingly, tumor invasion is one of the most important targets for designing treatments against metastastic cancers.

Among innovative strategies potentially useful for inhibiting cancer progression, therapy using cell-derived products, such as conditioned media, seems promising but has still not been soundly assessed.

Thus, Bar-Yehuda et al. (1999) Clin. Exp. Metastasis 17:531-535, following the observation that cancer rarely arose from skeletal muscle tissues, have shown that skeletal muscle cell conditioned medium administered to mice inoculated intravenously with melanoma or sarcoma cells, resulted in a statistically significant inhibition of metastatic lung foci. However, this treatment has not been further assessed in a clinical setting.

Besides, depending on the cell type, opposite results have been reported. In this regard, Chen et al. (2005) Surgery 138:382-90, in an attempt at determining how stromal microenvironment influences tumor progression, have shown that normal myofibroblasts or conditioned medium from normal myofibroblasts enhanced proliferation of colon cancer cells.

Gingival fibroblasts synthesise collagens (e.g. types I, III, V, VI, VII, XII), elastic fibers (oxytalan, elaunin and elastin), proteoglycans and glycosaminoglycans (e.g. decorin, biglycan), and glycoproteins (e.g. fibronectin, tenascin). Simultaneously, gingival fibroblasts synthesise enzymes that are able to degrade the macromolecular compounds (matrix metalloproteinases; MMPs), but also enzymes inhibiting active forms of MMPs (Inhibitors of metalloproteinases; TIMPs). Accordingly, gingival fibroblasts are important actors of extracellular matrix remodelling, either contributing to its synthesis or degradation.

### SUMMARY OF THE INVENTION

The present invention arises from the unexpected finding, by the inventors, that the conditioned medium of gingival fibroblasts inhibits malignant cell invasion ex vivo.

Thus, the present invention relates to a method for preventing or treating cancer in an individual, comprising administering the individual with a prophylactically or therapeutically effective quantity of a gingival fibroblast-derived product.

The present invention also relates to a gingival fibroblast-derived product for use in the prevention or treatment of cancer in an individual.

### DESCRIPTION OF THE FIGURES

FIG. 1 represents cell invasion of basal lamina extracts (vertical axis, in percentage) by HT1080 cells cultivated in IMDM medium with 10% FCS (HT1080 IMDM 10%), IMDM medium added with conditioned medium of human dermal fibroblasts cultivated with 10% FCS (HT1080 IMDM+DF 10%), IMDM medium added with conditioned medium of human gingival fibroblasts cultivated with 10% FCS (HT1080 IMDM+GF 10%).

FIG. 2 represents cell invasion of basal lamina extracts (vertical axis, in percentage) by M4T1 cells cultivated in IMDM medium with 10% FCS (M4T1 IMDM 10%), IMDM medium added with conditioned medium of human dermal fibroblasts cultivated with 10% FCS (M4T1 IMDM+DF 10%), IMDM medium added with conditioned medium of human gingival fibroblasts cultivated with 10% FCS (M4T1 IMDM+GF 10%).

FIG. 3 represents cell invasion of collagen I (vertical axis, in percentage) by HT1080 cells cultivated in IMDM medium with 10% FCS (HT1080 IMDM 10%),

IMDM medium added with conditioned medium of human dermal fibroblasts cultivated with 10% FCS (HT1080 IMDM+DF 10%), IMDM medium added with conditioned medium of human gingival fibroblasts cultivated with 10% FCS (HT1080 IMDM+GF 10%).

FIG. 4 represents cell invasion of collagen I (vertical axis, in percentage) by M4T1 cells cultivated in IMDM medium with 10% FCS (M4T1 IMDM 10%), IMDM medium added with conditioned medium of human dermal fibroblasts cultivated with 10% FCS (M4T1 IMDM+DF 10%), IMDM medium added with conditioned medium of human gingival fibroblasts cultivated with 10% FCS (M4T1 IMDM+GF 10%).

### DETAILED DESCRIPTION OF THE INVENTION

As intended herein the cancer to be prevented or treated according to the invention can be of any type. Preferably, it is a metastatic cancer or a cancer liable to form metastasis. Thus, preferably in the method of the invention, cancer cell invasion is inhibited. Furthermore, where the cancer forms tumors, in particular solid tumors, the method of the invention preferably prevents or treats tumor invasion. In other words, the method of the invention preferably prevents or treats metastasis of the cancer.

As intended herein the cancer preferably is a cancer of connective tissues. More preferably, the cancer is selected from the group consisting of breast cancer, and fibrosarcoma.

Preferably the individual is a mammal and more preferably a human.

Procedures for taking, culturing and preserving gingival fibroblasts are well known to the man skilled in the art and are particularly described in Naveau et al. (2006) J. Periodontol. 77:238-47 and in Gogly et al. (2007) Arterioscler. Thromb. Vasc. Biol. 27:1984-90.

Advantageously, gingival fibroblasts are easily sampled and cultured. Besides, gingival fibroblasts possess a high expansion rate.

Preferably, the gingival fibroblasts used in the method according to the invention are autologous, that is they are taken from the individual, to whom the gingival fibroblast-derived product is intended to be administered.

Advantageously, gingival fibroblasts provide for an almost limitless source of autologous fibroblasts. Furthermore, in case of aged skin, culture-competent autologous gingival fibroblasts are usually still available, whereas, in contrast, sources of culture-competent autologous dermal fibroblasts are scarce.

However, the gingival fibroblasts can also be allogenic, that is taken from another individual of the same species or heterologous, that is taken from another individual of another species.

As intended herein “gingival fibroblast-derived product” relates to any product which can be obtained from gingival fibroblasts in themselves or which contains gingival fibroblasts secretions. For example, it is preferred that the gingival fibroblast derived product is selected from the group consisting of gingival fibroblast whole cells, a gingival fibroblast culture, a gingival fibroblast extract, and a gingival fibroblast conditioned medium.

Gingival fibroblast extracts can be obtained by any cell fragmentation method known in the art.

Gingival fibroblast conditioned medium relates to any medium, such as a liquid cell culture medium, which has been contacted by gingival fibroblasts, in particular for a time sufficient for the gingival fibroblasts to have secreted in the medium.

Administration of the gingival fibroblast-derived product can proceed by any method known in the art. Thus, the gingival fibroblast-derived product can be injected locally, i.e. at a site near the tumor to be treated, or directly into the tumor to be treated. Besides, the gingival fibroblast-derived product can be administered by a route selected from the group consisting of the oral route, the subcutaneous route, the intravenous route, and the intramuscular route.

Preferably, the method according to the invention comprises the following steps:

taking gingival fibroblasts from the individual;
culturing the gingival fibroblasts;
obtaining a gingival fibroblast-derived product from the cultured gingival fibroblasts;
administering the gingival fibroblast-derived product to the individual.

### EXAMPLES

### Methods

### 1. Cell Culture

Malignant cells were obtained from the 4T1 cell line of murine breast carcinoma (M4T1) or from the HT1080 cell line of human fibrosarcome.

Conditioned medium is obtained after 24 hours of culture of 2 millions of gingival or dermal fibroblasts in 5 ml of Iscove's Modified Dulbecco's Medium (IMDM) (GIBCO ref:12440) with 10% of foetal calf serum (FCS) (GIBCO ref:1600-044) at 37° C. in 5% CO₂.

### 2. Cellular Invasion Test

60000 4T1 or HT1080 cells are cultivated with 10% FCS in a cellular invasion kit of basal lamina extracts (R&D system ref: 3455-96-K) or of type I collagen (R&D system ref: 3457-96-K) (50 μl) and optionally brought into contact with conditioned media obtained as described above (150 μl). Cellular invasion is measured after 24 hours according to the manufacturer instructions.

### Results

1. Conditioned medium from human gingival fibroblasts inhibit cellular invasion by malignant cells on basal lamina extracts

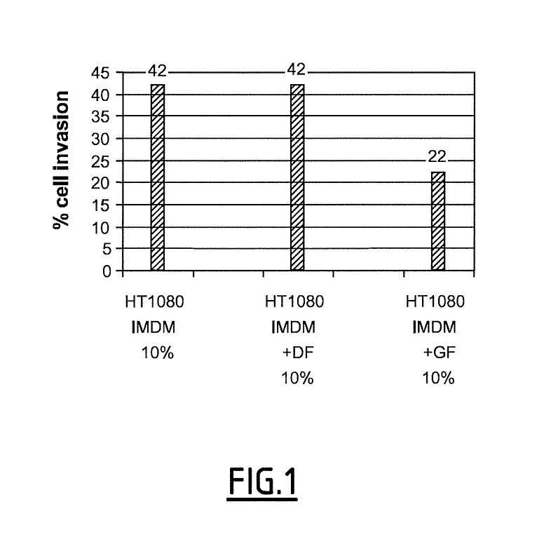

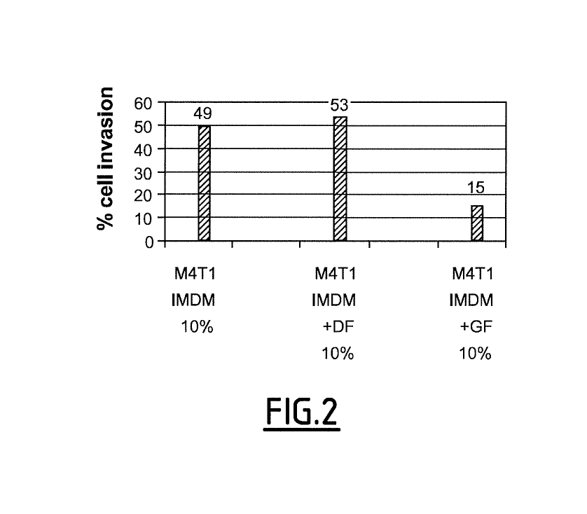

Similar results are obtained for the HT1080 and M4T1 malignant cells (FIGS. 1 and 2).

In the non-conditioned medium, cellular invasion is maximal (42% with HT1080 cells and 49% with M4T1 cells). In the medium conditioned by dermal fibroblasts, cellular invasion is of 42% for HT1080 cells and 53% for M4T1 cells. In the medium conditioned by gingival fibroblasts, cellular invasion is of 22% for HT1080 cells and 15% for M4T1 cells.

Thus, a medium conditioned by gingival fibroblasts inhibits cellular invasion of basal lamina extracts in comparison with a medium conditioned with dermal fibroblasts or with a non-conditioned medium.

2. Conditioned medium from human gingival fibroblasts inhibit cellular invasion by malignant cells in collagen I

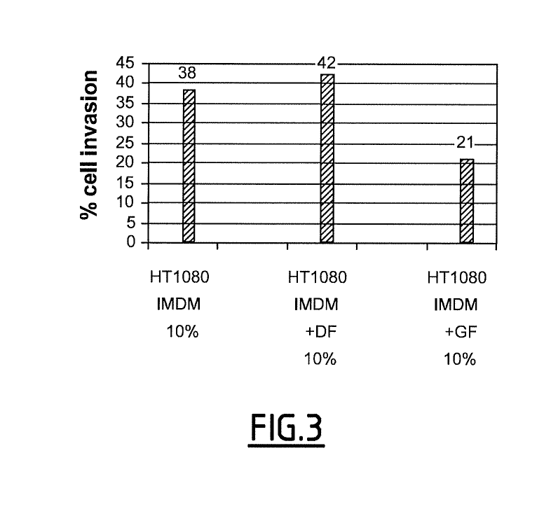

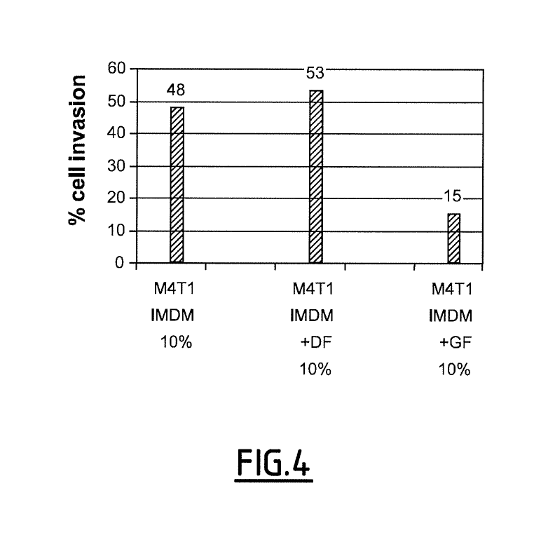

Similar results are obtained for the HT1080 and M4T1 malignant cells (FIGS. 3 and 4).

In the non-conditioned medium, cellular invasion is maximal (38% with HT1080 cells and 48% with M4T1 cells). In the medium conditioned by dermal fibroblasts, cellular invasion is of 42% for HT1080 cells and 53% for M4T1 cells. In the medium conditioned by gingival fibroblasts, cellular invasion is of 21% for HT1080 cells and 15% for M4T1 cells.

Thus, a medium conditioned by gingival fibroblasts inhibits cellular invasion of collagen I in comparison with a medium conditioned with dermal fibroblasts or with a non-conditioned medium.

All the cited references are incorporated herein by reference.

1. A method for treating metastatic cancer in an individual, comprising administering a therapeutically effective quantity of a gingival fibroblast-derived product to the individual, wherein cancer cell invasion is inhibited and wherein said gingival fibroblast-derived product is selected from the group consisting of gingival fibroblast whole cells, a gingival fibroblast culture, a gingival fibroblast extract and a gingival fibroblast conditioned medium.

2. The method according to claim 1, for treating tumor invasion.

3. The method according to claim 1, wherein the gingival fibroblast-derived product is administered by local injection.

4. The method according to claim 1, wherein the gingival fibroblast-derived product is administered by a route selected from the group consisting of the oral route, the subcutaneous route, the intravenous route, and the intramuscular route.

5. The method according to claim 1, wherein the gingival fibroblast-derived product is a gingival fibroblast conditioned medium.

6. The method according to claim 1, wherein the gingival fibroblast-derived product is obtained from gingival fibroblasts taken from the individual.

7. The method according to claim 1, wherein the cancer is a solid tumor.

8. The method according to claim 1, wherein metastasis of the cancer is treated.

9. The method according to claim 1, wherein the cancer is a cancer of connective tissues.
